(19) United States
(12) Patent Application Publication
(10) Pub. No.: US 2021/0121099 A1
(43) Pub. Date: Apr. 29, 2021

(54) ELECTRONIC VERTICAL JUMP MEASURING DEVICE

(71) Applicant: Matthew Wojcik, Hollis, NH (US)

(72) Inventor: Matthew Wojcik, Hollis, NH (US)

(21) Appl. No.: 16/667,881

(22) Filed: Oct. 29, 2019

(51) Int. Cl. A61B 5/11; G01S 17/88; G01S 7/481; A61B 5/00

(52) U.S. Cl. CPC A61B 5/1124; G01S 17/88; A61B 2503/10; A61B 5/742; A61B 5/002; G01S 7/481

(57) ABSTRACT

Using spindles that can be angularly displaced by the user through contact and a LiDAR system that measures the distance from the ground and the distance to the highest spindle contacted, a CPU calculates how high the user reached and how high the user jumped. The CPU displays the results on the display board and can use its Bluetooth interface to transmit results to any Bluetooth enabled device. The Bluetooth interface can also be used to reset the spindles using the reset mechanism.

### SOME BACKGROUND-PRIOR ART

### US PATENTS

| Patent Number | Kind Code | Issue Date | Patentee |
|---|---|---|---|
| 6,181,647 | B1 | Jan. 30, 2001 | Tipton, et al. |
| 6,167,356 | A | Dec. 26, 2000 | Squadron, et al. |
| 5,913,242 | B2 | Jun. 15, 1999 | Stussi; Edgar |
| 5,844,861 | A | Dec. 1, 1998 | Maurer; Gregory C. |
| 9,017,222 | B2 | Apr. 28, 2015 | Hofeldt; Robert |

Most vertical jump measurement devices use a very simple system to measure the vertical jump of the user. Spindles are stacked parallel to each other with a small gap between them. They are arranged in such away that they can only be angularly displaced along the axis that they are on. The highest spindle that is angularly displaced marks the highest spindle that the user was able to reach when they perform a jumping vertical reach. Knowing the height of the highest spindle the user displaced and the vertical reach of the user, he or she can calculate how high their vertical jump was.

### BRIEF SUMMARY OF THE INVENTION

In accordance with the main embodiment of the invention, there is disclosed an electromechanical device capable of measuring the height, vertical jump, and vertical reach of the user. The device displays the results on an LED display and transmits the results to any Bluetooth enabled device. The Bluetooth capability also allows electronic remote reset of the device.

### ADVANTAGES

This apparatus electronically measures the standing vertical reach, vertical jump, and height of the user which would otherwise require a tape measure or other separate measuring device. The device measures its own distance off the ground which would otherwise have to be measured by the user or be a known height off the ground such as when mounted to a pole of known length. The device can display the desired real time results of the user for bystanders to observe. The apparatus can transmit results and statistics to any Bluetooth enabled device and can be reset remotely by any Bluetooth enabled device. Prior attempts at measuring vertical reach are not able to measure vertical jump or the height of the user. Furthermore, measurement results of prior attempts require visual rather than electronic determination of results and are unable to electronically record or transmit those results.

### DRAWING-FIGURES

FIG. 1 shows an isometric view of the whole apparatus completely assembled. It is not connected to the pole that holds the apparatus upright for use.

FIG. 2 shows a detailed view of the spindle to highlight the unique holes and an exploded view of the spindle to understand how it is assembled.

FIG. 3 shows the apparatus stripped of its reset mechanism, spindles, and electronic components to get a better view of how those components are fixed to the main axle.

FIG. 4 shows the apparatus after the spindles have been displaced from their proper position by the user and how they interfere with the laser beam from the LiDAR system.

FIG. 5 shows a zoomed in top view of the apparatus as to better view the electronic components that the apparatus contains.

FIG. 6 shows the whole apparatus assembled and attached to a pole mechanism that stands the apparatus uptight to allow operation by the user.

FIG. 7 shows how a user may prepare to utilize the device and illustrates how the user's arm must be oriented to utilize the device.

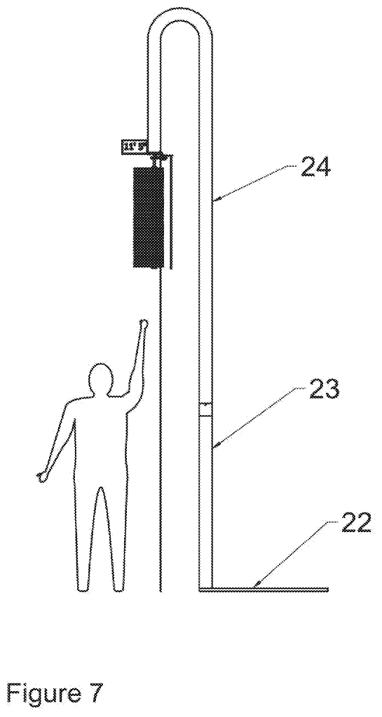

Drawings-Reference numerals

| | |
|---|---|
| Spindle | 1 |
| Reset Pole | 2 |
| LiDAR Laser Beam | 3 |
| LiDAR | 4 |
| Battery | 5 |
| Servo Motor | 6 |
| CPU | 7 |
| Display Board | 8 |
| Data Connection Wires | 9 |
| Main Axle | 10 |
| Axle Hole(in spindle) | 11 |
| Laser Hole(in spindle) | 12 |
| One Half of Spindle | 13 |
| Second Half of Spindle | 14 |
| Bolt(in spindle) | 15 |
| Nut(in spindle) | 16 |
| Washer | 17 |
| Battery Holder | 18 |
| Servo Motor Holder | 19 |
| LiDAR Holder | 20 |
| Mounting Holes | 21 |
| Platform | 22 |
| Base Pole | 23 |
| Hooked Pole | 24 |

### DETAILED DESCRIPTION

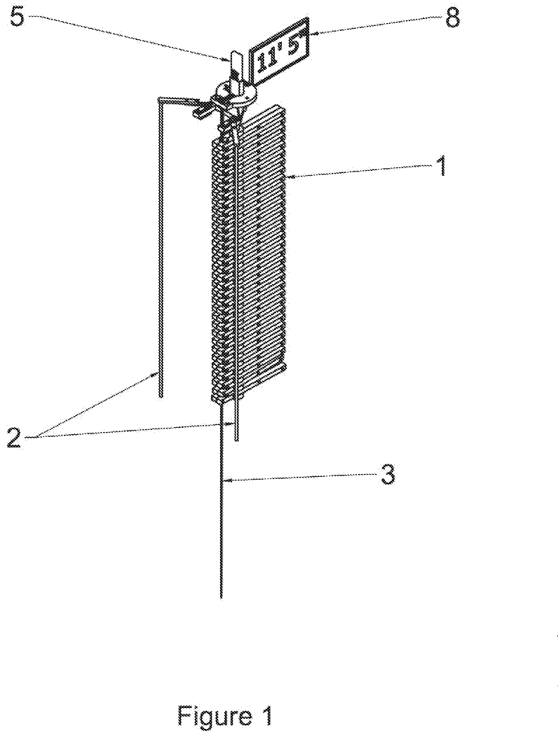

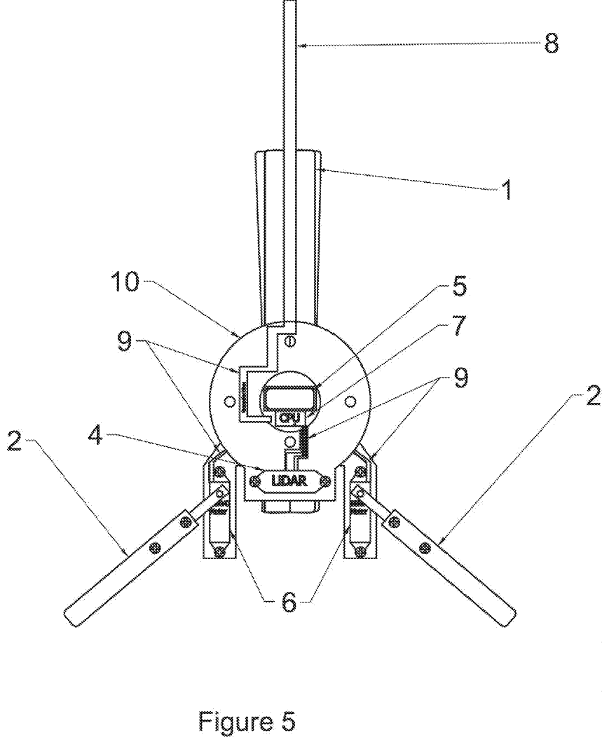

The apparatus measures the vertical jump of a user when the user jumps and displaces the spindles (item 1, FIGS. 1,4,5) on the device. The spindles (item 1), when displaced, are measured and recorded by the LiDAR system (item 4, FIG. 5) that is paired with a CPU (item 7, FIG. 5). The apparatus can then display the performance of the athlete on the display board (item 8, FIGS. 1,5) and it can send the results to an application on the user's Bluetooth enabled device.

### Central Processing Unit (CPU) (Item 7, FIG. 5)

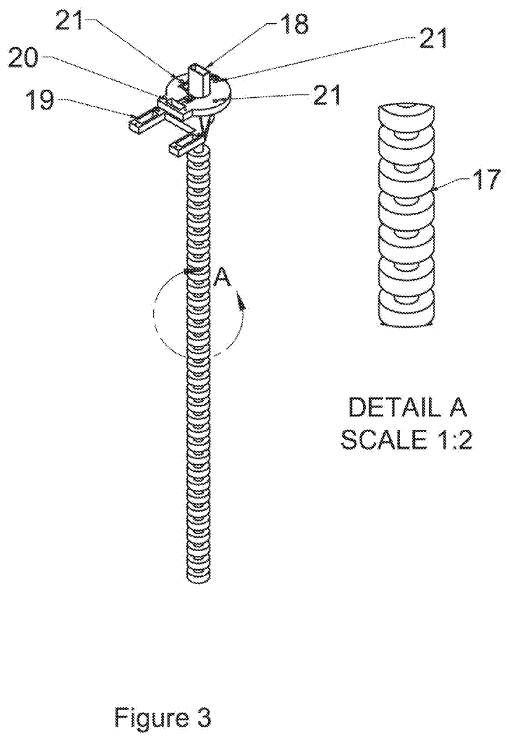

The CPU (item 7) will run the display board (item 8), LiDAR system (item 4) and reset mechanism via data connection wires (item 9). The CPU (item 7) itself, the display board (item 8), LiDAR system (item 4) and servo motors (item 7) all receive power from the battery pack (item 5, FIGS. 1,4,5) that is secured in the battery holder (item 18, FIG. 3). The CPU (item 7) will run calculations given specific data to determine the performance of the athlete. The CPU (item 7) is equipped with a transmitter and receiver which allows remote reset of the device and allows the performance of the user to be transmitted to any Bluetooth enabled device.

### Spindly: (Item 1)

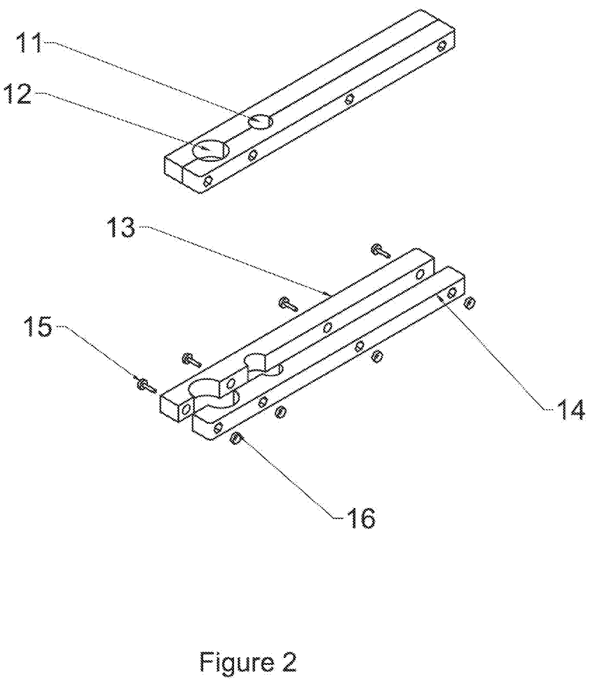

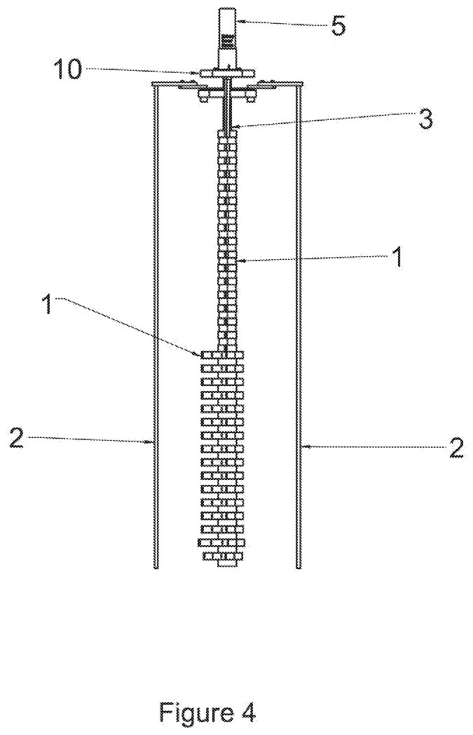

The spindles (item 1) are contacted when the user performs his or her jumping vertical reach. The spindles (item 1) have two holes: an axle hole (item 11, FIG. 2) and a laser hole (item 12, FIG. 2). The axle hole (item 11) is the hole that the axle (item 10, FIGS. 4,5) of the apparatus runs through. The laser hole (item 12) is the hole that the laser beam (item 3, FIGS. 1,4,6) from the LiDAR system (item 4) travels through. The spindles (item 1) consist of two halves (items 13, 14, FIG. 2) that are bolted together with nuts (item 16, FIG. 2) and bolts (item 15, FIG. 2). This allows for easy assembly of spindles (item 1) and allows for the washers (item 17, FIG. 3) to remain in their permanent positions between the spindles (item 1). The washers (item 17) remain in their permanent spots because the spindles' (item 1) axle hole (item 11) can be placed around the main axle (item 10, FIG. 5) of the apparatus and then bolted together. This will allow the spindles (item 1) to rotate on the main axle when contacted by the user while remaining a set distance away from each other along the main axle (item 10).

### Reset Mechanism

The reset mechanism realigns the spindles after they have been misaligned from being displaced by the user. The reset mechanism includes two servo motors (item 6, FIG. 5) that are secured in the servo holders (item 19, FIG. 3), located on each side of the spindles (item 1) with reset poles (item 2, FIGS. 1, 4, 5) that extend vertically downward and reach the bottom of the last spindle (item 1) on the apparatus. The reset mechanism is activated when the user uses the Bluetooth enabled device application to reset the spindles (item 1) after a user has used the apparatus and the spindles (item 1) are in misalignment. The servo motors (item 6), when prompted by the user via the application on the Bluetooth enabled device, will close the reset poles (item 2, FIGS. 1,4,5) simultaneously and contact the sides of the spindles (item 1) which will align them. When the spindles (item 1) are aligned, the laser hole (item 12) in the spindle (item 1) will allow the laser beam (item 3) from the LiDAR system (item 4) to pass through all of the spindles (item 1), touch the ground, and reflect back up to the LiDAR system (item 4).

### Detailed Operation

The apparatus has spindles (item 1, FIG. 5) that are restricted to angular movement around the main axle (item 10). The spindles (item 1) have two holes in them. One hole, the axle hole (item 11, FIG. 2), serves as the hole that the main axle (item 10) runs through to allow angular displacement of the spindle. The second hole, the laser hole (item 12), serves as the hole that the LiDAR system's (item 4) laser beam (item 3) goes through. The LiDAR system (item 4) is held sternly in place by the LiDAR holder (item 20, FIG. 3) that is built into the main axle (item 10) of the apparatus. When the spindles (item 1) are aligned in their proper position, the laser runs through every laser hole (item 12) in every spindle (item 1) that is on the apparatus. When a spindle (item 1) is displaced, the laser beam (item 3) will hit the outside edge of the laser hole (item 12). The laser beam (item 3) hitting the outside edge, will then reflect upward towards the LiDAR system (item 4) through the underside of all of the laser holes (12) of all of the spindles (item 1) above the contacted spindle (item 1). The CPU (item 7) will interpret what the LiDAR system's (item 4) measurement of distance is once every second. When the laser beam (item 3) hits the outside edge of the laser hole (item 12), the CPU (item 7) will read the output of the LIDAR system (item 4) and will calculate how far away the laser beam (item 3) was when it reflected. The laser beam (item 3) is able to return to the LIDAR system (item 4) because the spindles (item 1) above the spindle (item 1) that has been misaligned through jumper contact remain aligned to provide a clear path for the returning laser beam (item 3). With this information the CPU (item 7) will determine which spindle (item 1) was the highest displaced spindle. The CPU (item 7) will then run a calculation of the height of the LiDAR system from the ground minus the distance that was calculated when a spindle (item 1) was moved. This distance equals the height the user reached when the user performed a jumping vertical reach.

To determine standing reach, the user will stand under the apparatus and perform a vertical reach directly under the LiDAR system's (item 4) laser beam (item 3) causing the laser beam to reflect from the user's fingertip rather than the ground. The LiDAR system (item 4) measures how far off the ground it is minus the distance recorded when the user performed a vertical standing reach under the LiDAR system (item 4). This will result in the user's vertical standing reach height. The CPU (item 7), having stored both the standing vertical reach of the user and the jumping vertical reach of the user, can calculate the user's standing vertical jump distance (how high the user's feet traveled during the jump).

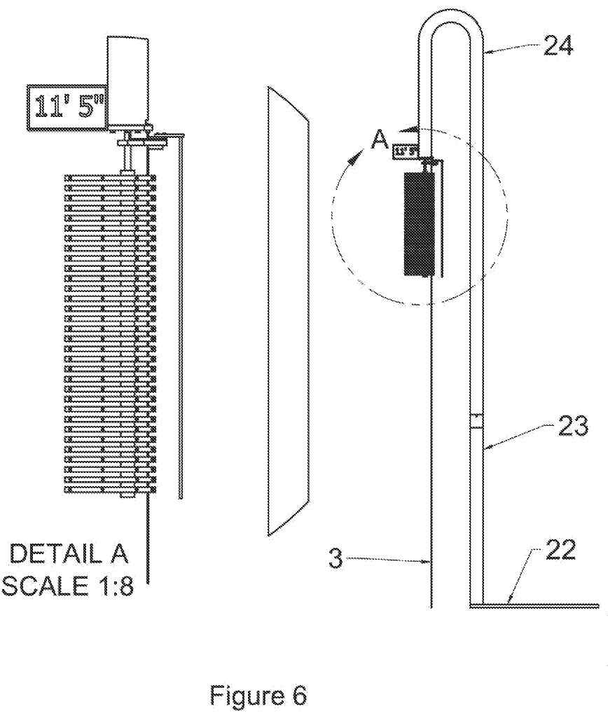

The entire apparatus described above must be mounted by the user at a height such that the user's jumping vertical reach does not reach or go beyond the top spindle (item 1). FIG. 6 shows a representation of how the apparatus can be mounted to a pole using bolts that run through the mounting holes (item 21, FIG. 3) and into threaded holes located on the hooked pole (item 24, FIGS. 6,7). The base pole (item 23, FIGS. 6,7) can be disconnected from the hook pole (item 24) for storage and to attach the apparatus to the hooked pole (item 24) which would be too high for most users to reach if it was attached to the base pole (item 23) and stood upright. The base pole (item 23) has a platform (item 22, FIGS. 6,7) that is used to counteract the weight of the apparatus. This platform (item 22, FIGS. 6,7) will have weights or objects on top of it that outweigh the apparatus in order to make it stable.

### Washers (Item 7)

The spindles (item 1) are separated from each other with washers (item 17) that are built into the main axle (item 10). These washers (item 17) serve as markers for where a spindles (item 1) must be located and provide an air gap between spindles (item 1) so that when a spindle is moved there is no friction between the spindles (item 1) that could potentially cause one spindle to be displaced that was never contacted by the user's hand. For example, without this barrier a spindle (item 1) above another spindle (item 1) that was contacted could be moved by the friction between the two spindles (item 1) and result in an erroneous reading of the user's jumping vertical reach.

### Bluetooth Enabled Device Application

The Bluetooth enabled device that is paired with the apparatus will be able to send and receive signals via Bluetooth to and from the apparatus. The apparatus will be able to send data about the performance of the user to this device. This will allow the user to log and track his or her performance over time. The application will also allow for a remote control reset of the spindles (item 1) using the reset mechanism. This will allow the spindles (item 1) to be reset in proper formation thereby allowing the laser beam (item 3) to pass through all of the laser holes (item 12) in all of the spindles (item 1) and reflect from the ground below it. The application will also have the ability to control what the display board (item 8) reads when a user performs a standing vertical jump. The application can control the display board (item 8) to display the vertical jumping reach, vertical jump, or height of the user. The main purpose for the display board (item 8) is for bystanders as well as the user to view the user's performance. This can be used if a bystander does not have a coupled Bluetooth enabled device with which to view the performance of the user.

1. The device can electronically measure the vertical jump of the user
The device can electronically measure the jumping vertical reach of the user
The device can electronically measure the vertical reach of the user
The device can electronically measure its distance from the ground to calibrate itself for other measurements.
The device can electronically read which spindles were contacted by the user.
The device can display the data requested by the user.
The device can transmit results and statistics to any Bluetooth enabled device.
The device can reset its spindles remotely via command from any Bluetooth enabled device.
